(19) United States
(12) Patent Application Publication
(10) Pub. No.: US 2023/0159959 A1
(43) Pub. Date: May 25, 2023

(54) BACILLUS SP. PRODUCING BIOFLOCCULANT AND BIOSURFACTANT AND USE THEREOF

(71) Applicant: ZHEJIANG UNIVERSITY, Hangzhou (CN)

(72) Inventors: Chunfang ZHANG, Hangzhou (CN); Feng ZENG, Hangzhou (CN); Hanghai ZHOU, Hangzhou (CN); Lijia JIANG, Hangzhou (CN); Dongdong ZHANG, Hangzhou (CN); Yanhong LI, Hangzhou (CN); Shichen LI, Hangzhou (CN)

(21) Appl. No.: 18/097,461

(22) Filed: Jan. 16, 2023

Related U.S. Application Data
(63) Continuation of application No. PCT/CN2021/100071, filed on Jun. 15, 2021.

(51) Int. Cl. C12P 1/04; C02F 3/34; C12N 1/20

(52) U.S. Cl. CPC C12P 1/04; C02F 3/34; C12N 1/20; C02F 2103/10

(57) ABSTRACT

The present application discloses a Bacillus sp. producing a bioflocculant and a biosurfactant. The microbial classification of the Bacillus sp. is named Bacillus sp. SS15, which has been preserved in China Center for Type Culture Collection on Mar. 29, 2021, and its preservation number is CCTCC M2021295; the 16S rRNA sequence of SS15 is shown as SEQ ID NO.1. Bacillus sp. SS15 obtained by the present application has the function of simultaneously producing a bioflocculant and a biosurfactant. The bioflocculant and biosurfactant produced by Bacillus sp. SS15 of the present application not only have high activity, but also show strong tolerance in the ranges of pH (2-12), temperature (4° C.-100° C.) and salinity (0-100 g/L). Meanwhile, they can be applied to the remediation of a fracturing flowback liquid and can effectively promote the removal of chroma, suspended solids, COD, n-alkanes and polycyclic aromatic hydrocarbons at the same time.

### REFERENCE TO SEQUENCE LISTING

The present application is being filed along with a Sequence Listing in electronic format. The Sequence Listing is provided as a file entitled DF222187US-SEQUENCE LISTING ST.26, created Dec. 22, 2022, which is approximately 4 Kb in size. The information in the electronic format of the Sequence Listing is incorporated herein by reference in its entirety.

### TECHNICAL FIELD

The present application relates to the field of microorganisms, in particular to a Bacillus sp. producing a bioflocculants and a biosurfactant and use thereof in the remediation of a fracturing flowback fluid.

### BACKGROUND

A fracturing flowback fluid is the oilfield wastewater produced when hydraulic fracturing technology is used to exploit oil and gas fields. The wastewater contains petroleum, dissolved solids, suspended solids (SSs), chemicals and other pollutants, so effective treatment is required before subsequent disposal. Because the fracturing flowback fluid has the characteristics of a high COD, a high suspended solids content, a high viscosity and complex composition, the existing technologies are mainly physical and chemical processes such as coagulation, air flotation and activated carbon adsorption. However, the above physical processes have the disadvantages of a high treatment cost and a limited pollutant removal capacity; the use of chemical bioflocculants such as polyacrylamide has the disadvantages of a high cost and secondary pollution to the environment. A biological treatment process uses microorganisms to absorb the organic substances in wastewater and decompose them into substances with lower toxicity or non-toxicity. Compared with the physical and chemical treatment processes, the biological treatment process has the advantages of environmental friendliness, no secondary pollution, a low cost and thorough degradation. However, at present, there is little research on the treatment of suspended solids, COD and hydrocarbons in the fracturing flowback fluid by microbial technology. The main reasons may be as follows: first, the suspended solids in fracturing flowback fluid are relatively stable, and the use of the bioflocculant is limited because of its unstable flocculation activity; second, the composition of the fracturing flowback fluid is complex, and its environment (pH, salinity, toxic chemicals, etc.) is harsh, so that microbial growth and metabolism are easily restricted; third, the fracturing flowback fluid contains a large amount of hydrophobic organic matter, and the low bioavailability would result in a limited biodegradation treatment effect.

### SUMMARY

In order to solve the above technical problems, the present application provides a Bacillus sp. producing a bioflocculant and a biosurfactant and use thereof in fracturing flowback fluid treatment. The obtained Bacillus sp. SS15 has the functions of producing a bioflocculant and a biosurfactant and degrading hydrocarbons at the same time. The bioflocculant and biosurfactant produced by the Bacillus sp. SS15 have high activity, and the two products show strong tolerance in a wide range of pH (2-12), temperature (4° C.-100° C.) and salinity (0 g/L-100 g/L).

The specific technical solution of the present application is as follows:

In one aspect, the present application provides a Bacillus sp. producing a bioflocculant and a biosurfactant, wherein the microbial classification of the Bacillus sp. is named Bacillus sp. SS15, which was deposited in China Center for Type Culture Collection on Mar. 29, 2021, with a preservation number of CCTCC M2021295; the 16S rRNA sequence of SS15 is shown as SEQ ID NO.1.

The Bacillus sp. SS15 of the present application comes from the oil-polluted intertidal sediments near Xincheng Bridge in Zhoushan City. Through further experiments, it is found that the surfactants produced by SS15 are phospholipids, and the critical micelle concentration is 44.37 mg/L, which can reduce the surface tension of water from 72 mN/m to 36.56 mN/m, and its properties can maintain strong stability in different ranges of pH, temperature and NaCl concentration. The produced bioflocculant contains polysaccharide and protein, and its flocculation efficiency for kaolin suspension is 84.91%, and its performance can be stable at different pH, temperatures and NaCl concentrations.

It can be seen from the above that the Bacillus sp. SS15 can produce a biosurfactant and a bioflocculant by itself, and compared with synthetic surfactants, the biosurfactant produced by the Bacillus sp. ss 15 has better biodegradability, low toxicity, high efficiency, low critical micelle concentration and other characteristics; compared with chemical bioflocculants, the produced bioflocculant has better biodegradability, low toxicity and high efficiency. Meanwhile, the bioflocculant and biosurfactant produced by the Bacillus sp. SS15 can effectively promote the removal of chroma (85.72%), suspended solids (94.40%), COD (84.86%), n-alkanes (49.95%) and polycyclic aromatic hydrocarbons (66.46%).

The Bacillus sp. of the present application also includes cultures or passaged cultures of SS15.

In a second aspect, the present application provides a fermentation method of the Bacillus sp. SS15. The specific fermentation conditions are as follows: olive oil is used as a carbon source, yeast extract and urea are used as nitrogen sources, the fermentation temperature is 25° C.-37° C., the salinity is 0 g/L-10 g/L, and the pH is 6.8-7.2.

In a third aspect, a concentration of the olive oil is preferably 0.8-1.2 g/L, a concentration of the yeast extract is 3.5 g/L, and a concentration of the urea is 0.5 g/L.

In a fourth aspect, the Bacillus sp. SS15 and its produced bioflocculant as well as biosurfactant can be applied to the remediation of a fracturing flowback fluid.

Compared with the prior art, the present application has the advantages that:

(1) A strain SS15, which produces a bioflocculant and a biosurfactant, was isolated from the oil-polluted intertidal sediments near Xincheng Bridge in Zhoushan City, and identified as Bacillus sp. by 16s rRNA. Furthermore, it was found that the biosurfactants produced by SS15 were phospholipids, and the critical micelle concentration was 44.37 mg/L, which could reduce the surface tension of water from 72 mN/m to 36.56 mN/m, and their properties were stable in the ranges of pH (2-12), temperature (4° C.-100° C.) and salinity (0 g/L-100 g/L). The flocculating efficiency of the produced bioflocculant to a kaolin suspension (5 g/L) is 84.91%, and its performance is stable in the ranges of pH (2-12), temperature (4° C.-100° C.) and salinity (0 g/L-100 g/L), showing very strong tolerance.

(2) Through TLC, FTIR and GC-MS analysis, the biosurfactant is characterized as a phospholipid, and the bioflocculant contains saccharide and protein. The addition of Bacillus sp. SS15 and its bioflocculant and biosurfactant can effectively promote the removal of the chroma (85.72%), suspended solids (94.40%), COD (84.86%), n-alkanes (49.95%) and polycyclic aromatic hydrocarbons (66.46%) of the fracturing flowback fluid.

(3) The Bacillus sp. SS15 of the present application originates from petroleum hydrocarbon polluted sediments, and has good environmental adaptability, so it can well promote the removal of COD and hydrocarbons in the fracturing flowback fluid.

(4) The use of SS15 in the remediation of the fracturing flowback fluid shows that it can effectively remove chroma, SS, COD, hydrocarbons and the like at the same time. By combining SS15 with the bioflocculant and biosurfactant produced by SS15, that is, firstly adding a fermentation broth of Bacillus sp. and the biosurfactant produced by Bacillus sp. for degradation, and then adding the bioflocculant produced by Bacillus sp. for flocculation, the effect can be improved to a great extent, wherein the chroma removal rate is up to 85.72%, SS is reduced from 1090 mg/L to 61 mg/L, COD is reduced from 8371 mg/L to 1267 mg/L, n-alkanes are reduced from 860.7 mg/L to 430.8 mg/L and polycyclic aromatic hydrocarbons are reduced from 1161.2 μg/L to 379.6 μg/L.

### BRIEF DESCRIPTION OF DRAWINGS

FIG. 1 shows the test results of the performances of SS15, including, the appearance of the strain on the plate (a), an oil drain ring experiment (b), a droplet collapse experiment (d), an emulsifiability index E₂₄ (c), and phylogenetic tree (e).

FIG. 2 shows the experimental results of the flocculation characteristics of the bioflocculant produced by SS15 for kaolin, including the dosage of the bioflocculant (a), the type of cation (b), the cation concentration (c), pH (d), and the settling time (e).

FIG. 3 shows the performance test results of the bioflocculant produced by SS15, that is, the stability of the bioflocculant under different pH, temperatures and NaCl concentrations.

FIG. 4 is the characterization of the bioflocculant produced by SS15, including the SEM micrograph (a) and the FTIR spectrum (b).

FIG. 5 shows the performance test results of the biosurfactant produced by SS15, including the CMC measurement (a), and the stability of the biosurfactant under different pH, temperatures and NaCl concentrations.

FIG. 6 is the characteristics of the biosurfactant produced by SS15, including the TLC chromatogram (a), the FTIR spectrum (b), and the GC-MS chromatogram (c).

FIG. 7 shows the optimization results of treatment conditions of the fracturing flowback fluid by the bioflocculant produced by SS15, including the dosage of the bioflocculant (a), the type of cation (b), the cation concentration (c), the pH (d), the settling time (e), and the flocculation effect diagram (f).

FIG. 8 shows the removal effect of SS15 and its bioflocculant and biosurfactant on COD.

FIG. 9 shows the removal effect of SS15 and its bioflocculant and biosurfactant on chroma, SS and COD, including the removal efficiency of chroma under different experimental groups (a), the content of SS and the corresponding removal efficiency in different experimental groups (b), and the content of COD and the corresponding removal efficiency in different experimental groups (c).

FIG. 10 shows the removal effect of SS15 and its bioflocculant and biosurfactant on petroleum hydrocarbon, including the concentrations of total n-alkanes and n-alkanes with different chain lengths in different experimental groups (a), and the concentrations of total polycyclic aromatic hydrocarbons in different experimental groups (b).

### DESCRIPTION OF EMBODIMENTS

The present application provides a Bacillus sp. SS15, whose microbial classification is named Bacillus sp. SS15, which was preserved in China Center for Type Culture Collection on Mar. 29, 2021, and its preservation number is CCTCC M2021295; the 16S rRNA sequence of SS15 is shown as SEQ ID NO.1.

The present application provides cultures or passaged cultures of SS15.

The present application provides a fermentation method of the Bacillus sp. SS15. The fermentation conditions are as follows: olive oil is used as a carbon source, yeast extract and urea are used as nitrogen sources, the fermentation temperature is 25° C.-37° C., the salinity is 0-10 g/L, and the pH is 6.8-7.2. Preferably, the concentration of the olive oil is 0.8-1.2 g/L, the concentration of the yeast extract is 3.5 g/L, and the concentration of the urea is 0.5 g/L.

The present application provides use of the Bacillus sp. SS15 and its bioflocculant and biosurfactant in the remediation of a fracturing flowback fluid.

The present application will be further described with reference to the following examples.

### Example 1 Isolation of Strain SS15

Strain isolation sample was collected from the intertidal beach polluted by oil near Xincheng Bridge in Zhoushan, Zhejiang Province. 1 g of sediment was added into saline solution and mixed homogeneously, the supernatant was then dipped to draw lines on agar medium for bioflocculant isolation. After culturing at 30° C. for 12-48 h, single colony was selected individually for further separation and purification. The components of the bioflocculant isolation medium were glucose (10 g/L), yeast extract (3.5 g/L), urea (0.5 g/L), K₂HPO₄ (5 g/L), KH₂PO₄ (2 g/L), NaCl (0.1 g/L) and MgSO₄ (0.5 g/L). After activation, the selected single colony was inoculated into a 250 mL conical flask filled with 100 mL of the bioflocculant isolation medium, and cultured at 30° C. and 180 rpm for 3 days. Subsequently, 1 mL of a fermentation broth was used to evaluate the bioflocculant-producing potential based on the flocculation activity Then, the strains with biosurfactant production ability were selected from the above strains with bioflocculant-producing potential. Specifically, each bacterial solution was inoculated into a 250 mL conical flask containing 100 mL of the biosurfactant isolation medium, and cultured at 30° C. and 180 rpm for 3 days. The components of the biosurfactant isolation medium were olive oil (1 g/L), yeast extract (3.5 g/L), urea (0.5 g/L), K₂HPO₄ (5 g/L), KH₂PO₄ (2 g/L), NaCl (0.1 g/L) and MgSO₄ (0.5 g/L). After cultivation, a proper amount of the fermentation broth was collected and centrifuged at 8000 rpm for 10 min to obtain cell-free supernatant, i.e., a bacteria-removed fermentation broth. Whether bacteria have the ability to produce a biosurfactant was judged by surface tension. Then, the bacteria-removed fermentation broth that contain a biosurfactant was collected for a performance test:

1. Oil Spreading Experiment

100 mL of ultrapure water was added into a clean 22 cm dish, and 400 μL of light crude oil was added dropwise into the water. The crude oil would spread rapidly on the surface of the water. When it was stable, 10 μL of bacteria-removed fermentation broth was slowly added dropwise with a pipette; the diameter of the oil spreading was measured, and the ultrapure water with an equal volume was added dropwise as a negative control.

2. Droplet Collapse Experiment

50 μL of cell-free fermentation supernatant after centrifugation was added dropwise to the parafilm, then the shape and the spread of the droplets were observed. Afterwards, 2 μL of methylene blue staining solution (1 wt %, which has no effect on the shape of the droplets) was added to the water droplets for dyeing and photographing. The diameter of the droplets was measured with a ruler, and an equal volume of uninoculated fermentation medium stained with methylene blue was added dropwise as a negative control.

3. Measurement of the Surface Tension and Emulsification Index E₂₄ of the Fermentation Broth

The surface tension of the fermentation broth was measured at room temperature using a surface tensiometer (BZY-201, Shanghai Fangrui Instrument Co. Ltd., China).

3 mL of the bacteria-removed fermentation broth and 3 mL of olive oil were added into the test tube, mixed completely with ultrasonication for 10 min, and allowed to stand at room temperature for 24 h, and the emulsification index: E₂₄(%)=(height of emulsion layer/total height of liquid)×100% was determined.

4. Measurement of the Flocculation Activity of the Fermentation Broth.

1 mL of the bacteria-removed fermentation broth was added to 25 mL of a kaolin suspension containing 5 g/L and 1 mL of CaCl₂ solution (10 g/L), vortexed for 3 min, and was allowed to stand for 10 min. The liquid 2 cm below the liquid level was collected to evaluate the flocculation efficiency, the flocculation efficiency (%)=(A−B)/A×100%, where A is the OD₅₅₀ absorbance of the negative control and B is the OD₅₅₀ absorbance of the sample.

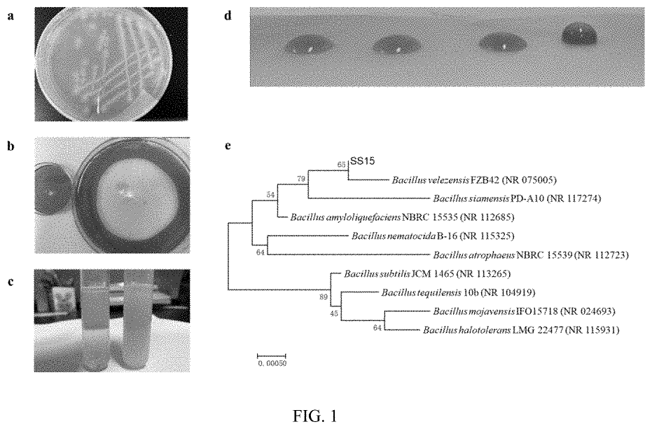

Based on the above performance test, the strain with the highest biosurfactant production capacity was selected and named as SS15, its image is shown in FIG. 1(a), and its oil spreading results are shown in FIG. 1(b). The diameter of the oil spreading of the fermentation broth is 13.6 cm. The experimental results of droplet collapse are shown in FIG. 1(d), with three experimental groups (0.7±0.01 cm in diameter) from left to right, and the right one as the control (0.5 cm in diameter). The result of emulsifying olive oil with the biosurfactant produced by SS15 strain is shown in FIG. 1(c), and the emulsifying index E₂₄ is 52%. The flocculation efficiency of the fermentation broth (i.e., the supernatant) is 77.97%.

### Example 2 Identification of Strain SS15

Molecular identification of SS15 strain was carried out. Easy Pure Bacteria Genomic DNA Kit (Transgene Biotechnology Co, ltd., Beijing, China) was used to extract DNA. The obtained DNA was used as a template DNA for polymerase chain reaction (PCR), and the forward primer and the reverse primer used for PCR amplification were 27f and 1492r, respectively. 16s rRNA gene amplicon was sequenced (TSINGKE Biotechnology Co, ltd., Hangzhou, China), and the results were compared by a basic local alignment search tool (https://blast.ncbi.nlm.nih.gov/). MEGA 7.0 software (Pennsylvania State University, State College, Pa., USA) was used to construct a phylogenetic tree. As shown in FIG. 1(e), the strain SS15 and Bacillus velezensis FZB42 belong to the same cluster on the phylogenetic tree, so it can be determined that they belong to Bacillus sp. The strain was deposited in China Center for Type Culture Collection on Mar. 29, 2021, with the preservation number of CCTCC M 2021295, and the microbial classification was named Bacillus sp.;

### Example 3 Extraction and Performance Identification of Bioflocculant

3.1 Extraction of Bioflocculant

A strain SS15 was cultured in a fermentation medium containing 1% olive oil as a carbon source for 1 day. The fermentation medium was as follows: the concentration of olive oil was 1 g/L, the concentration of yeast powder was 3.5 g/L, and the concentration of urea was 0.5 g/L, the fermentation temperature was 25° C.-37° C. and the pH was 6.8-7.2. The culture solution was centrifuged at 8000 rpm for 25 min to collect the supernatant. Pre-cooled absolute ethanol (4° C.) with twice volume was added into the supernatant; the solution was allowed to stand overnight at 4° C., centrifuged at 9000 rpm for 30 min; the precipitate was collected, and then rinsed with a small amount of ultrapure water for 2-3 times, and the precipitate obtained was the crude bioflocculant.

3.2 Performance Evaluation of the Bioflocculant

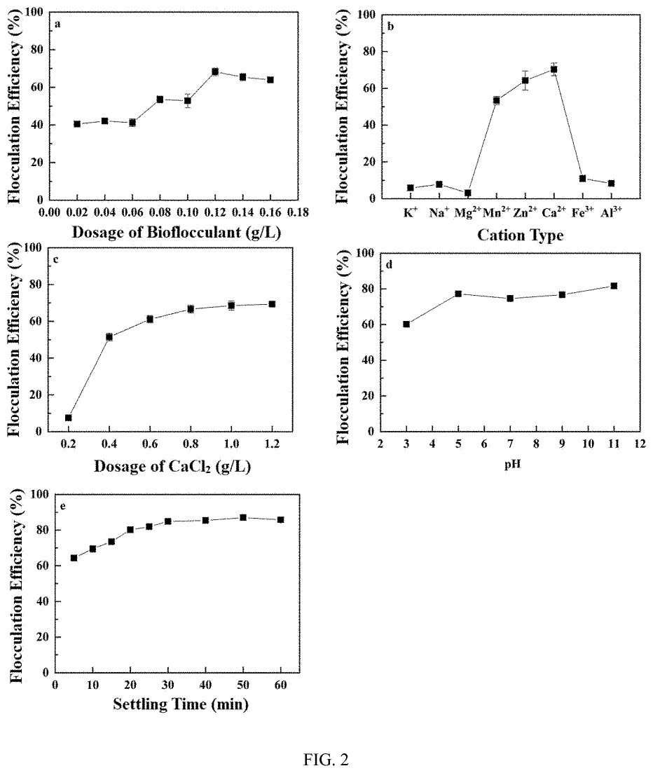

(1) Experiment of bioflocculant dosage: 1 mL of bioflocculant aqueous solutions with different concentrations (0.5-4.0 g/L) was added to 23 mL 5 g/L kaolin suspension, respectively. Then, 1 mL of a CaCl₂ solution (10 g/L) was added, vortexed for 3 min and allowed to stand for 10 min, the liquid 2 cm below the liquid level was collected to evaluate the flocculation efficiency. The flocculation efficiency (%)=(A−B)/A×100%, where A was the OD₅₅₀ absorption value of the negative control, and B as the OD₅₅₀ absorption value of the sample. As shown in FIG. 2(a), a lower dosage of the bioflocculant (0.12 g/L) could show a good flocculation effect on kaolin suspension.

(2) Experiment of cationic coagulant aid: 1 mL of 10 g/L CaCl₂, MgCl₂, NaCl, KCl, FeCl₃, AlCl₃ were respectively added to 23 mL of a 5 g/L kaolin suspension and 1 mL of a bioflocculant solution (optimum concentration: 0.12 g/L) was added; the mixture was vortexed for 3 min, then allowed to stand for 10 min, and the liquid 2 cm below the liquid level was collected to evaluate the flocculation efficiency; the experimental results are shown in FIG. 2(b). Compared with monovalent and trivalent cationic coagulant aids, bivalent cationic coagulant aids have better flocculation promoting effect. The optimal cation (CaCl₂) was prepared into different concentration solutions and flocculation experiments were carried out. The results in FIG. 2(c) show that CaCl₂ solution with a low concentration (1.0 g/L) can exhibit better flocculation efficiency.

(3) pH of the reaction system: a kaolin suspension was mixed with 6 M hydrochloric acid and 1 M NaOH solution to different pH values (2-12). The results of flocculation efficiency under different pH conditions are shown in FIG. 2(d). The bioflocculant has a good flocculation effect on the kaolin suspension with different pH values (3-11).

(4) Different flocculation settling time (5-60 min) were set, and the flocculation efficiency of the bioflocculant under different settling time periods was measured. As shown in FIG. 2(e), the bioflocculant can achieve a good flocculation effect in a short settling time (30 min).

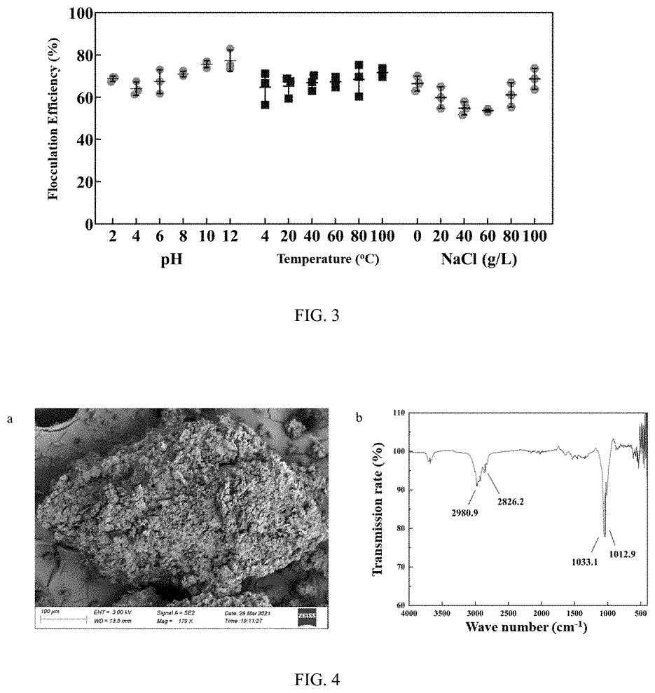

(5) Stability test: the bioflocculant was prepared into 3 g/L solution with sterile pure water for the following experiments. Temperature stability: the bioflocculant aqueous solution was treated at different temperatures (4° C.-100° C.) respectively, and the flocculation efficiency of the solution after 1 hour treatment at each temperature was measured; pH stability experiment: the bioflocculant solution was adjusted to different pH values (2-12) with 6 M hydrochloric acid and 1 M NaOH solution, and the flocculation efficiencies of bioflocculant solution at different pH values were measured; stability of salinity: NaCl of different amount were added to the prepared bioflocculant aqueous solution to make the solution with salinity in the range of 0-100 g/L, and the flocculation efficiencies of bioflocculant solution at different salinities were measured. Wherein, in the flocculation experiment, the dosage of the bioflocculant was 0.12 g/L and CaCl₂ was 1.0 g/L. As shown in FIG. 3, the bioflocculant showed strong flocculation activity in the wide ranges of pH (2-12), temperature (4° C.-100° C.) and salinity (0-100 g/L).

3.3 SEM

A scanning electron microscope (Sigma 500) was used to analyze the surface microstructure of the bioflocculant. The SEM results of the bioflocculant were shown in FIG. 4(a). The rough surface of the bioflocculant is beneficial for combining a large amount of suspended solids and precipitation thereof.

3.4 FTIR

In order to identify the chemical bond types of the bioflocculant, a FTIR spectrometer (CCR-1, Thermo-Nicolet, America) was used for Fourier transform infrared spectrum analysis in the spectral range of 4000 cm⁻¹−400 cm⁻¹. The FTIR results of the bioflocculant were shown in FIG. 4(b). A weak CH tensile vibration band was observed at 926.2 cm⁻¹, which may be caused by carbohydrate derivatives. The strong absorption peak at 1033.1 cm⁻¹ was —C—O—C— stretching vibration, which indicated that the bioflocculant contained carboxylic acid, hydroxyl and methoxy, and the methoxy group was identified as a sugar derivative group.

3.5 Component Identification

(1) Quantitative analysis of polysaccharide: the quantitative analysis is a phenol-sulfuric acid method. 0, 0.4, 0.6, 0.8, 1.0, 1.2, 1.4, 1.6 and 1.8 mL of a glucose standard solution (0.1 mg/L) were added into 25 mL colorimetric tubes, distilled water was then added to bring the volume to 2 mL. Subsequently, 1.0 mL of a phenol solution (6%, v/v) and 5 mL of concentrated sulfuric acid were added, and were allowed to stand for 20 min; after cooling, the absorbance value was detected with a spectrophotometer at 490 nm, and the standard curve was established with the blank solution as the reference. Finally, 1 mL of the bioflocculant solution was taken to determine the polysaccharide content via the above phenol-sulfuric acid method, and the polysaccharide content was calculated based on the established standard curve.

(2) Quantitative analysis of protein: the quantitative method was a Coomassie brilliant blue method. Bovine serum albumin was prepared to 1 mg/L standard solution. 0.01 g of coomassie brilliant blue was dissolved in 5 mL of 90 vol % ethanol and 12 mL of 85% (v/v) phosphoric acid, which was diluted to 100 ml with distilled water to prepare a coomassie brilliant blue solution. 0.02, 0.04, 0.06, 0.08, 0.10 mL of the standard solution of bovine serum albumin were added into 25 mL colorimetric tubes; distilled water was added to 0.10 mL; 5 mL of coomassie brilliant blue was added, and the OD value at 595 nm was measured after 2 min to obtain the standard curve of protein. 1 mL of the bioflocculant solution was taken to determine the protein content via the above Coomassie brilliant blue method, and the content of protein in the sample was calculated according to the standard curve.

The results show that the crude bioflocculant contained polysaccharide (5.95%) and protein (8.29%).

### Example 4 Extraction and Performance Evaluation of Biosurfactant

4.1 Extraction of Biosurfactant

A strain SS15 was cultured in a fermentation medium containing olive oil as a carbon source for 3 days. The fermentation medium was as follows: the concentration of olive oil was 1.0 wt %, the concentration of yeast extract was 3.5 g/L, and the concentration of urea was 0.5 g/L. The fermentation temperature was 25° C.-37° C. and the pH was 6.8-7.2. The culture solution was centrifuged at 8000 rpm for 25 min to collect supernatant. The supernatant was extracted with ethyl acetate for three times. The extracts were combined and concentrated by a rotary evaporator to obtain a biosurfactant.

4.2 Determination of Critical Micelle Concentration (CMC)

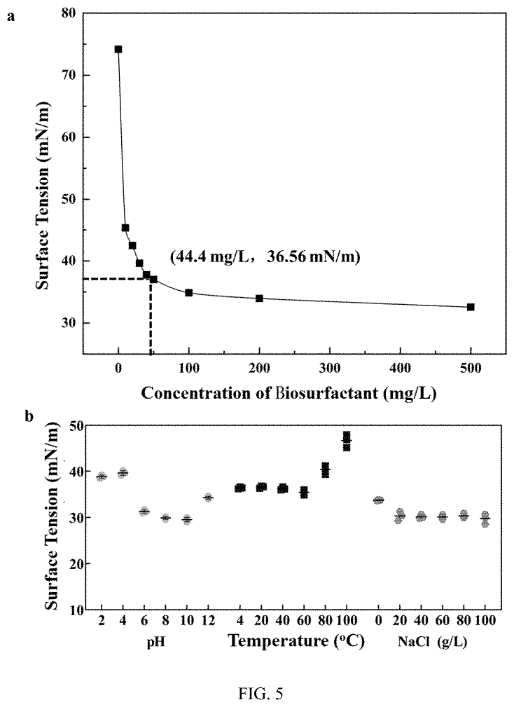

The extracted biosurfactant was prepared into a series of biosurfactant solutions with different concentrations, and the surface tension of the solutions was measured by a surface tensiometer. The surface tension of the solution would decrease with the increase of the biosurfactant concentration. When the surface tension of the solution tended to be stable and no longer decreased, the corresponding concentration was seen as the critical micelle concentration of the biosurfactant. As shown in FIG. 5(a), the CMC of the biosurfactant produced by SS15 was 44.4 mg/L, and the corresponding surface tension was 36.56 mN/m.

4.3 Determination of Biosurfactant Stability

Stability to temperature: a biosurfactant solution with a concentration of 44.37 mg/L(CMC) was prepared, the solution was treated at different temperatures (4° C.-100° C.) for 30 min, and the surface tension of the solution after each temperature treatment was detected; stability to pH: the biosurfactant solution was adjusted to different pH values (2-12) with 6 M of hydrochloric acid and 1 M of a NaOH solution, and the surface tensions of the solution at different pH values were measured; stability to salinity: different amounts of NaCl were added to the prepared biosurfactant solution to make the solution with the salinity in the range of 0-100 g/L, and the surface tensions of the biosurfactant solution at different salinities were measured. The specific results were shown in FIG. 5(b). The extracted biosurfactant showed strong tolerance in the ranges of pH (2-12), temperature (4° C.-100° C.) and salinity (0-100 g/L).

4.4 Thin Layer Chromatography (TLC)

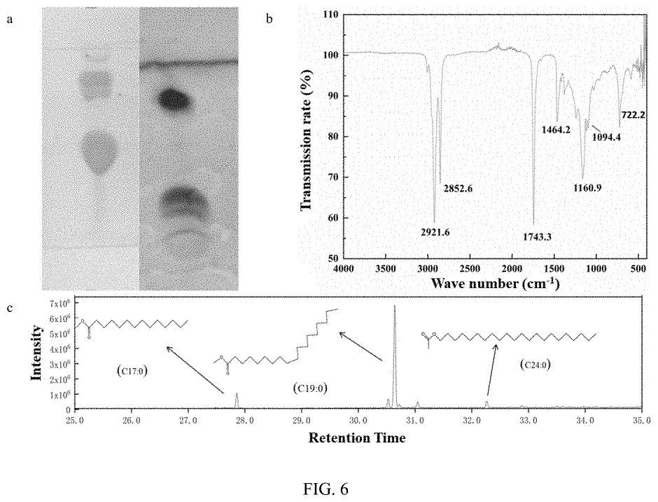

50 mg of the biosurfactant was dissolved in 5 mL of methanol, and about 10 μL of the solution was spotted on a silica gel plate (Marine Biotech Co, Qingdao, China). Petroleum ether/n-hexane (4:1, v:v) was used as a mobile phase to separate compounds. A phosphomolybdic acid-ethanol reagent (5 g phosphomolybdic acid mixed with 50 mL absolute ethanol) was used to detect phospholipid surfactants, and the positive results showed pale blue spots. A silicone plate was treated with iodine vapor, and the lipid showed yellow spots. FIG. 6(a) shows the TLC diagram of the biosurfactant produced by SS15. The phosphomolybdic acid-ethanol solution was sprayed on the A plate to detect phospholipids, and the light blue color indicated positive (right side); the B plate was tested for lipid by iodine vapor coloration, and lemon yellow indicated positive (left side).

4.5 FTIR

To identify the chemical bond type of the biosurfactant, Fourier transform infrared spectroscopy (FTIR) was used in the spectral range of 4000 cm⁻¹−400 cm⁻¹ by a FTIR spectrometer (CCR-1, Thermo-Nicolet, America). The FTIR results were shown in FIG. 6(b). The bands at 2921.6 cm⁻¹, 2852.6 cm⁻¹ and 1464.2-1160.9 cm⁻¹ were the characteristics of the stretching vibration of the fatty chain (—CH₃, —CH₂—). A strong absorption peak at 743.3 cm⁻¹ indicated that it had ester bonds and carboxylic acid groups (—COOR and —COOH). The weak absorption peak at 1464.2 cm⁻¹ was caused by the bending vibration of C—H on the carbon chain, which indicated the existence of a carbon chain structure. The absorption peak at 800-500 cm⁻¹ might be caused by methylene shearing vibration of the bacterial protein. In addition, the absorption peak at 1094.4 cm⁻¹ might be caused by the P—O—C chain.

4.6 Analysis of Fatty Acid Composition by Gas Chromatography-Mass Spectrometry (GC-MS)

To verify the fatty acid composition of the surfactant produced by strain SS15, the biosurfactant produced by strain SS15 was hydrolyzed and methylated, then extracted and concentrated with n-hexane, and then analyzed by GC-MS. 10 mg of the extracted biosurfactant with 5 mL of 2 M hydrochloric acid-methanol (1:1, v/v) was added into the ampoule, and the ampoule was flushed with nitrogen and sealed. The mixture was reacted in a water bath at 100° C. for 4 h, and extraction was then implemented twice with 2 mL n-hexane. The extracts were combined and diluted 100 times in a centrifuge tube with a plug for GC-MS analysis. The instrument was Shimadzu QP2020 GC-MS, the carrier gas was helium; and the column flow rate was 1.5 mL/min. The inlet temperature was 260° C.; the gas interface temperature was 260° C., the initial column temperature was 60° C.; and the temperature was raised to 260° C. at a heating rate of 5° C./min, and kept for 10 min. Mass spectrometry conditions: the ion source temperature was 200° C., the scanning range was 50-500 amu, the injection volume was 1 μL, and the shunt ratio was 50:1. In order to estimate the possible fatty acid composition of the biosurfactant, the GC-MS results of structural comparison of fatty acid methyl ester were searched in the National Institute of Standards and Technology (NIST) mass spectrometry database. The results of the GC-MS analysis were shown in FIG. 6(c).

The results of TLC and FTIR showed that the biosurfactants produced by SS15 were phospholipids. After methyl esterification, the fatty acids of the biosurfactants were heptadecanoic acid, nonadecanoic acid and tetracosanoic acid as analyzed by GC-MS.

### Example 5 Use in Fracturing Flowback Fluid Remediation

To test whether Bacillus sp. SS15 and its bioflocculant and biosurfactant can effectively remove the chroma, SS, COD, n-alkanes and polycyclic aromatic hydrocarbons from a fracturing flowback fluid through flocculation and biodegradation, SS15 and its bioflocculant and biosurfactant were added into a fracturing flowback fluid, and the corresponding removal effect on chroma, SS, COD, n-alkanes and polycyclic aromatic hydrocarbons through flocculation and biodegradation was evaluated. Wherein, the determination of chroma and SS was carried out by using the national standard. After the cultivation, the culture solution was centrifuged at 8000 rpm for 10 min to remove bacteria cells. The COD was determined by using a dichromate method. The remaining oil was extracted and analyzed by GC-MS to determine the degradation efficiency of different treatment groups. In short, 3 mL of the culture solution was added with an equal volume of n-hexane to recover the residual oil in the culture and the extraction was repeated three times, the upper organic phases were combined and dried with anhydrous Na₂SO₄. Finally, the n-hexane phase was diluted 10 times, and the components of C8-C40 were quantified by GC-MS (QP2020, Shimadzu) equipped with SH Rxi-5Sil MS column (30 m×0.25 μm×0.25 mm, Shimadzu). Helium was used as a carrier gas with a flow rate of 1.2 mL/min. The temperature parameters of the column oven were set as follows: the initial temperature was set at 50° C., which was kept for 2 min; and the temperature was raised to 300° C. at the rate of 6° C./minute, which was kept for 25 min. The ion source and interface temperatures were set at 230 and 300° C., respectively. The collection mode was set to a selected ion monitoring mode, and the ion of each component corresponded to the retention time of the external standard (34 alkanes). In addition, according to (EPA)-PAHs specified by the U.S. Environmental Protection Agency, the remaining PAHs in the culture solution were extracted and analyzed.

All data were the average of 3 replicates. In order to test the significant differences of SS and COD among different treatments, SPSS 19.0 software (IBM Corp, USA) was used for a t-test analysis. The error bar indicated the standard deviation. * meant that the difference between the original sample and other treatments was significant (*P<0.05) or very significant (**P<0.01).

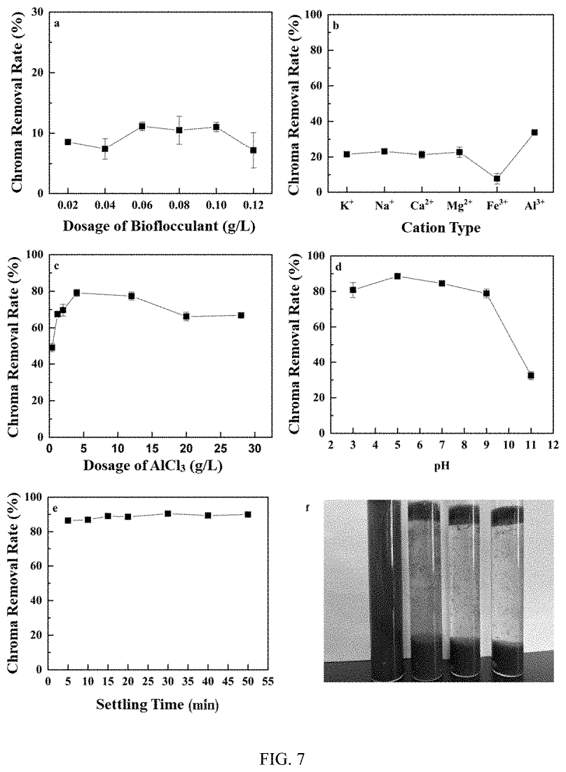

For the flocculation experiment, the chroma removal efficiency was taken as the consideration index and the flocculation treatment conditions of the bioflocculant for the fracturing flowback fluid (from Ji 7 oilfield in Xinjiang) were optimized by investigating the factors including the dosage of the bioflocculant, the cation type, the cation concentration, the pH of mixed system as well as the flocculation settling time. FIG. 7 shows the optimization of treatment conditions of the bioflocculant for the fracturing flowback fluid. The results showed that the best flocculation conditions were 0.06 g/L bioflocculant, 4 g/L AlCl₃, pH=5, and settling time of 30 min. The treatment effect of the fracturing flowback fluid is shown in FIG. 7(f).

A pre-biodegradation experiment took the COD removal rate as an index to study the effect of different additives on the bioremediation of the fracturing flowback fluid. In short, the biodegradation includes five different treatment groups, namely control, biostimulation 1, biostimulation 2, bioaugmentation 1 and bioaugmentation 2. For all the five different treatment groups, 250 mL conical bottles containing 100 mL of a fracturing flowback fluid were used, wherein 1 mL of a yeast extract solution (YE, 10 g/L) was added to biostimulation 1(Bs1); 1 mL of the biosurfactant (5 g/L) produced by strain SS15 was added to biostimulation 2(Bs2); 1 mL of a SS15 bacterial solution was added to bioaugmentation 1(Ba1) (which was centrifuged and then re-suspended with an equal volume of saline solution); 1 mL of a SS15 bacterial solution, 1 mL of YE and 1 mL of the biosurfactant (5 g/L) produced by the strain SS15 were added into bioaugmentation 2(Ba2); 3 mL of sterile ultrapure water was added to the control group. All supplements were added three days before cultivation (biosurfactant added in all treatment groups was only added once on the first day), and the supplement systems were all 3 mL (sterile ultrapure water was added if the volume was less than 3 mL). In addition, Origin is the original group without any treatment. Samples from all treatment groups were prepared in triplicate.

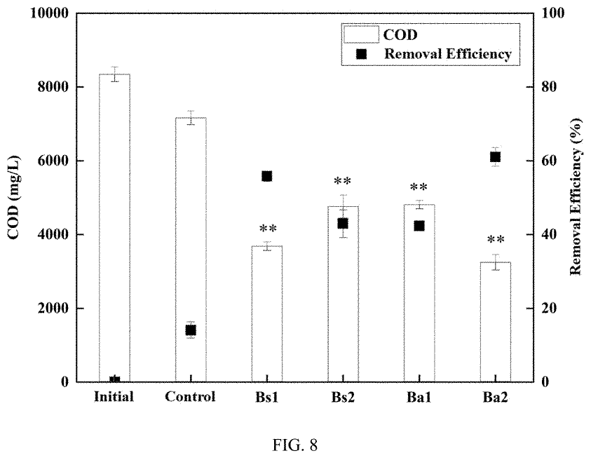

FIG. 8 shows the influence of different additives on the biodegradation of fracturing flowback fluid. Among them, the COD removal efficiency of the control, Bs1, Bs2, Ba1, Ba2 and other treatment groups were 14.11%, 55.83%, 42.94%, 42.33% and 61.04%, respectively. The results showed that the best COD removal effect could be obtained by adding 1 mL of the 10 g/L yeast extract (YE), 1 mL of the SS15 bacteria solution and 1 mL of the biosurfactant (5 g/L) on the first day of cultivation.

By combining the results of the flocculation experiment and biodegradation pre-experiment, the remediation effect of the strain SS15 and its bioflocculant and biosurfactant on the fracturing flowback fluid through flocculation and biodegradation was studied. All experiments were carried out in a 250 mL conical flask containing 100 mL of the fracturing flowback fluid. The following groups were set: for the control (degradation) group, 3 mL sterile ultrapure water was added before the cultivation; for the flocculation group, the optimized flocculation conditions (adding 0.06 g/L bioflocculant and 4 g/L AlCl₃, settling for 30 min after flocculation treatment) were used for flocculation experiment; for the degradation group, the optimized degradation conditions (adding 1 mL of 10 g/L yeast extract, SS15 bacteria solution, and 5 g/L biosurfactant, respectively) were used. The flocculation+degradation group was subjected to a biodegradation experiment after flocculation treatment; the degradation+flocculation group was treated by flocculation after biodegradation, and the flocculation conditions used in the experiment and the nutrients added in the culture were the same as those used in the flocculation+degradation group, i.e., both the groups adopted optimized conditions. All experimental groups (except the flocculation group) were cultured in shaking at 30° C. and 180 rpm for 7 days.

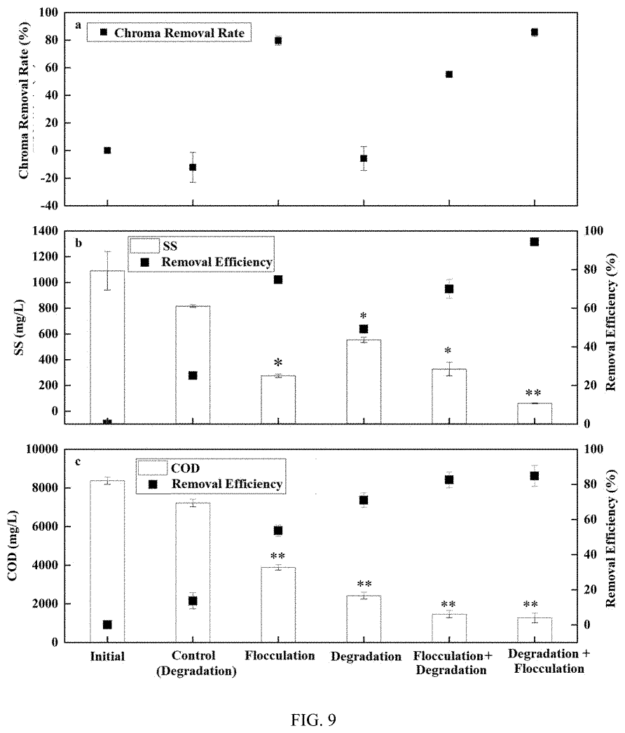

FIG. 9(a) shows the chroma removal efficiencies of different experimental groups. The chroma removal efficiencies of the control (degradation) group, the flocculation group, the degradation group, the flocculation+degradation group and the degradation+flocculation group were −12.17%, 79.66%, −5.75%, 55.16% and 85.72% respectively. The results showed that the bioflocculant could effectively reduce the chroma of the fracturing flowback fluid.

FIG. 9(b) shows the contents of suspended solids and corresponding removal efficiencies of different experimental groups. Among them, the original sample: 1090 mg/L; the control (degradation) group: 816 mg/L (reduction by 25.14%); the flocculation group: 275 mg/L (reduction by 74.80%); the degradation group: 553.5 mg/L (reduction by 49.22%); the flocculation+degradation group: 327 mg/L (reduction by 70.00%); the degradation+flocculation group: 61 mg/L (reduction by 94.40%). The results showed that the bioflocculant can effectively remove the suspended solids in fracturing flowback fluid.

FIG. 9(c) shows the COD concentration and the corresponding removal efficiencies of different experimental groups. The initial COD content was 8371 mg/L; the control (degradation) group was 7219 mg/L (reduction by 13.76%); the flocculation group was 3878 mg/L (reduction by 53.67%); the degradation group was 2419 mg/L (reduction by 71.10%); the flocculation+degradation group was 1459 mg/L (reduction by 82.57%); the degradation+flocculation group was 1267 mg/L (reduction by 84.86%). The results showed that the bioflocculant could effectively remove COD; the inoculated strain SS15 and its biosurfactant could effectively remove COD; and the combination of the flocculation and degradation experiments could effectively remove the COD of the fracturing flowback fluid.

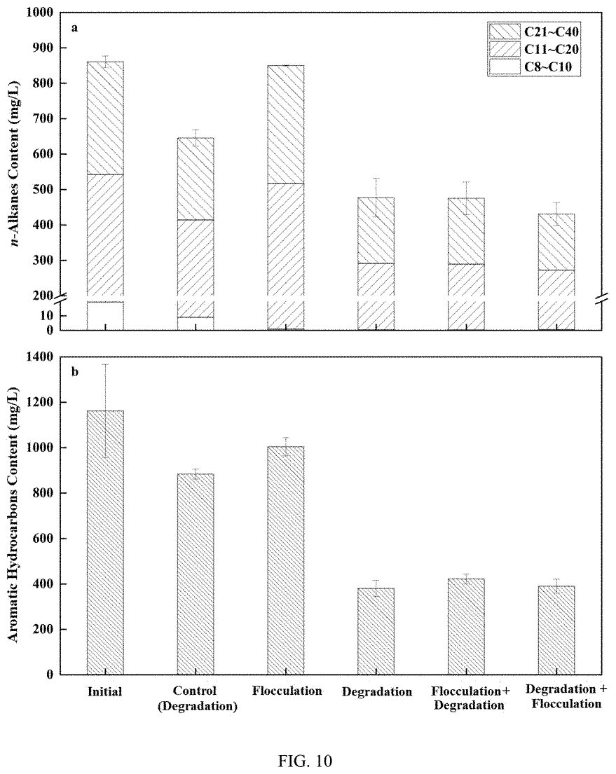

FIG. 10(a) shows the concentrations of total n-alkanes and n-alkanes with different chain lengths under different treatments. The initial content of the total n-alkanes (C8-C40) was 861 mg/L; the contents of n-alkanes (C8-C40) in the control (degradation) group, the flocculation group, the degradation group, the flocculation+degradation group and the degradation+flocculation group were 646 mg/L (reduction by 24.99%), 846 mg/L (reduction by 1.78%), 477 mg/L (reduction by 44.57%), 475 mg/L (reduction by 44.80%) and 431 mg/L (reduction by 49.95%), respectively. The results showed that the addition of the strain SS15 and its biosurfactant could effectively promote the removal of normal paraffin from fracturing flowback fluid.

FIG. 10(b) shows the concentration of the total polycyclic aromatic hydrocarbons under different treatments. The initial content of total polycyclic aromatic hydrocarbons was 1161 μg/L; the contents of polycyclic aromatic hydrocarbons in the control (degradation) group, the flocculation group, the degradation group, the flocculation+degradation group and the degradation+flocculation group were 883 μg/L (reduction by 23.92%), 1004 μg/L (reduction by 13.57%), 380 μg/L (reduction by 67.31%), 421 μg/L (reduction by 63.73%) and 389 μg/L (reduction by 66.46%), respectively. The results showed that the addition of the strain SS15 and its biosurfactant could effectively promote the removal of polycyclic aromatic hydrocarbons in the fracturing flowback fluid.

To sum up, the flocculating efficiency of the bioflocculant produced by Bacillus sp. SS15 to kaolin suspension was 84.91%, and it showed strong tolerance in different ranges of pH (2-12), temperature (4° C.-100° C.) and salinity (0-100 g/L).

The CMC of the biosurfactant produced by Bacillus sp. SS15 was 44.37 mg/L, and it showed strong tolerance in different ranges of pH (2-12), temperature (4° C.-100° C.) and salinity (0-100 g/L).

Through TLC, FTIR and GC-MS analysis, the biosurfactants were characterized as phospholipids. The results of polysaccharide quantification, protein quantification and FTIR analysis showed that the bioflocculant contained polysaccharide and protein. The addition of Bacillus sp. SS15 and its bioflocculant and biosurfactant with the combination of flocculation and biological treatments could effectively promote the removal of color, SS, COD and hydrocarbons (including n-alkanes and PAHs) in the fracturing flowback fluid.

In addition, SS15 and its bioflocculant and biosurfactant could effectively remove chroma, SS, COD and hydrocarbons (including n-alkanes and PAHs) after being added into the fracturing flowback fluid. This study proved that inoculating SS15 and adding the bioflocculant and biosurfactant produced by SS15 was an effective method to remediate the fracturing flowback fluid.

Unless otherwise specified, the raw materials and equipment used in the present application are common raw materials and equipment in the field. Unless otherwise specified, the methods used in the present application are conventional methods in this field.

The above description is only the preferred embodiments of the present application, and is not intended to limit the present application. Any simple modifications, changes and equivalent changes made to the above embodiments according to the technical essence of the present application still belong to the protection scope of the technical solution of the present application.

SEQ ID NO. 1
atctggtaaccttcggcggctggctccataaaggtt
acctcaccgacttcgggtgttacaaactctcgtgg
tgtgacgggcggtgtgtacaaggcccgggaacgta
ttcaccgcggcatgctgatccgcgattactagcga
ttccagcttcacgcagtcgagttgcagactgcgat
ccgaactgagaacagatttgtgggattggcttaac
ctcgcggtttcgctgccctttgttctgtccattgt
agcacgtgtgtagcccaggtcataaggggcatgat
gatttgacgtcatccccaccttcctccggtttgtc
accggcagtcaccttagagtgcccaactgaatgct
ggcaactaagatcaagggttgcgctcgttgcggga
cttaacccaacatctcacgacacgagctgacgaca
accatgcaccacctgtcactctgcccccgaagggg
acgtcctatctctaggattgtcagaggatgtcaag
acctggtaaggttcttcgcgttgcttcgaattaaa
ccacatgctccaccgcttgtgcgggcccccgtcaa
ttcctttgagtttcagtcttgcgaccgtactcccc
aggcggagtgcttaatgcgttagctgcagcactaa
ggggcggaaaccccctaacacttagcactcatcgt
ttacggcgtggactaccagggtatctaatcctgtt
cgctccccacgctttcgctcctcagcgtcagttac
agaccagagagtcgccttcgccactggtgttcctc
cacatctctacgcatttcaccgctacacgtggaat
tccactctcctcttctgcactcaagttccccagtt
tccaatgaccctccccggttgagccgggggctttc
acatcagacttaagaaaccgcctgcgagcccttta
cgcccaataattccggacaacgcttgccacctacg
tattaccgcggctgctggcacgtagttagccgtgg
ctttctggttaggtaccgtcaaggtgccgccctat
ttgaacggcacttgttcttccctaacaacagagct
ttacgatccgaaaaccttcatcactcacgcggcgt
tgctccgtcagactttcgtccattgcggaagattc
cctactgctgcctcccgtaggagtctgggccgtgt
ctcagtcccagtgtggccgatcaccctctcaggtc
ggctacgcatcgtcgccttggtgagccgttacctc
accaactagctaatgcgccgcgggtccatctgtaa
gtggtagccgaagccaccttttatgtctgaaccat
gcggttcagacaaccatccggtattagccccggtt
tcccggagttatcccagtcttacaggcaggttacc
cacgtgttactcacccgtccgccgctaacatcagg
gagcaagctcccatctgtccgctcgactgcagtat
agcacccgccccgg

What is claimed is:

1. A Bacillus sp. producing a bioflocculant and a biosurfactant, wherein the microbial classification of the Bacillus sp. is named Bacillus sp. SS15, which was deposited in China Center for Type Culture Collection on Mar. 29, 2021, with a preservation number of CCTCC M2021295; the 16S rRNA sequence of SS15 is shown as SEQ ID NO.1.

2. A fermentation method of the Bacillus sp. according to claim 1, wherein fermentation conditions are as follows: olive oil is used as carbon source, yeast powder and urea are used as nitrogen sources, a fermentation temperature is 25° C.-37° C., a NaCl concentration is 0 g/L-10 g/L, and a pH is 6.8-7.2.

3. The fermentation method according to claim 2, wherein a concentration of the olive oil is 0.8 g/L-1.2 g/L, a concentration of the yeast powder is 3.5 g/L, and a concentration of the urea is 0.5 g/L.

4. Use of the Bacillus sp. according to claim 1, the bioflocculant and the biosurfactant produced by the Bacillus sp. in the remediation of a fracturing flowback fluid.

5. The use according to claim 4, wherein the remediation of the fracturing flowback fluid comprises: removal of the chroma, SS, COD and hydrocarbons of the fracturing flowback fluid.

6. The use according to claim 5, wherein the hydrocarbons comprise n-alkanes and polycyclic aromatic hydrocarbons (PAHs).

7. The use according to claim 4, comprising
adding a fermentation broth of the Bacillus sp. and the biosurfactant produced by the Bacillus sp. into the fracturing flowback liquid for degradation, and adding the bioflocculant produced by the Bacillus sp. for flocculation.


SEQUENCE LISTING (1 sequence)

SEQ ID NO: 1 - DNA, 1450 bp, Bacillus sp.
  source 1..1450: mol_type = genomic DNA
atctggtaaccttcggcggctggctccataaaggttacctcaccgacttcgggtgttacaaactctcgtggtgtgacgggcggtgtgtacaaggcccgggaacgtattcaccgcggcatgctgatccgcgattactagcgattccagcttcacgcagtcgagttgcagactgcgatccgaactgagaacagatttgtgggattggcttaacctcgcggtttcgctgccctttgttctgtccattgtagcacgtgtgtagcccaggtcataaggggcatgatgatttgacgtcatccccaccttcctccggtttgtcaccggcagtcaccttagagtgcccaactgaatgctggcaactaagatcaagggttgcgctcgttgcgggacttaacccaacatctcacgacacgagctgacgacaaccatgcaccacctgtcactctgcccccgaaggggacgtcctatctctaggattgtcagaggatgtcaagacctggtaaggttcttcgcgttgcttcgaattaaaccacatgctccaccgcttgtgcgggcccccgtcaattcctttgagtttcagtcttgcgaccgtactccccaggcggagtgcttaatgcgttagctgcagcactaaggggcggaaaccccctaacacttagcactcatcgtttacggcgtggactaccagggtatctaatcctgttcgctccccacgctttcgctcctcagcgtcagttacagaccagagagtcgccttcgccactggtgttcctccacatctctacgcatttcaccgctacacgtggaattccactctcctcttctgcactcaagttccccagtttccaatgaccctccccggttgagccgggggctttcacatcagacttaagaaaccgcctgcgagccctttacgcccaataattccggacaacgcttgccacctacgtattaccgcggctgctggcacgtagttagccgtggctttctggttaggtaccgtcaaggtgccgccctatttgaacggcacttgttcttccctaacaacagagctttacgatccgaaaaccttcatcactcacgcggcgttgctccgtcagactttcgtccattgcggaagattccctactgctgcctcccgtaggagtctgggccgtgtctcagtcccagtgtggccgatcaccctctcaggtcggctacgcatcgtcgccttggtgagccgttacctcaccaactagctaatgcgccgcgggtccatctgtaagtggtagccgaagccaccttttatgtctgaaccatgcggttcagacaaccatccggtattagccccggtttcccggagttatcccagtcttacaggcaggttacccacgtgttactcacccgtccgccgctaacatcagggagcaagctcccatctgtccgctcgactgcagtatagcacccgccccgg